(19) United States
(12) Patent Application Publication
(10) Pub. No.: US 2007/0060823 A1
(43) Pub. Date: Mar. 15, 2007

(54) Internally vented multi-function esophageal gastric tube

(76) Inventors: Guy M. Giroux, Topeka, KS (US); Jeffery W. Glasgow, Lawrence, KS (US)

Correspondence Address: SHUGHART THOMSON & KILROY, PC, 120 WEST 12TH STREET, KANSAS CITY, MO, 64105, US

(21) Appl. No.: 11/207,690

(22) Filed: Aug. 19, 2005

(51) Int. Cl. A61B 5/02

(52) U.S. Cl. 600/485

(57) ABSTRACT

An esophageal gastric tube includes lumens for suction, venting and auscultation. The auscultation lumen serves as a conduit for electrical conductors to a thermistor positioned distally of the lumen for measuring core temperature without occlusion of the lumen. A stethoscope membrane encircles the tube, forming a sound chamber. Suction and vent lumens extend from the proximal tube end to the distal tip, while the stethoscope lumen terminates at the sound chamber distal end, for reducing suction noise interference with auscultation. A longitudinal slot in the tubular sidewall enables effective communication between the sound chamber and the stethoscope lumen. The stethoscope membrane includes internal circumferential ribs preventing occlusion of the slot and permitting 360° auscultation. The tube distal sidewall is apertured into the suction lumen, enabling suctioning of the gastric contents. Vent and suction lumens communicate within the distal end of the tube to provide an internal vent for relieving negative stomach pressure.

### BACKGROUND OF THE INVENTION

The present invention is broadly concerned with a tubular apparatus for insertion through the nose or mouth and into the stomach of a patient for simultaneously detecting heart and lung sounds, monitoring core body temperature, and providing suction drainage and gastric pressure equalization. More particularly, it is concerned with a multiluminate esophageal gastric tube having a temperature sensor and stethoscope, a suction lumen and a vent lumen with an internal distal opening.

In surgical procedures performed on patients under general anesthesia, an esophageal stethoscope is generally introduced into the esophagus in order to enable continuous monitoring of the patient's heart and breath sounds. The device may also include thermistor structure for monitoring the patient's core body temperature. Such monitoring is necessary in order to promptly detect any variations from the normal which may occur during the procedure, as well as to guide therapies such as warming, cooling and certain pharmacological interventions for rare conditions such as malignant hyperthermia.

Esophageal stethoscope structure includes a membrane sleeve that surrounds the perimeter of the catheter to form a sound chamber. The sound chamber extends 360° around the catheter and communicates the sounds pneumatically with an interior lumen by means of a series of apertures in the sidewall of the catheter. During use, the membrane is positioned in the area of the esophagus adjacent the mediastinum for picking up and magnifying body sounds in the area of the heart and lungs. In order to function effectively as a diaphragm, this membrane must necessarily be constructed of a thin, flexible material. In use, the membrane is easily pushed down by the esophageal tissue and collapsed against the outer sidewall of the catheter and the apertures. Where a nasogastric tube is employed, it may exert lateral pressure against the membrane. Collapse of one or more portions of the membrane sleeve against the catheter sidewall reduces the 360° directional range of the sound transmission, while collapse of the membrane sleeve onto the apertures muffles the clarity of sound transmission into the stethoscope lumen. Either type of contact pressure on the membrane sleeve serves to compromise the efficiency of the stethoscope.

These conventional esophageal stethoscope devices are not well-suited to remain in place for an extended period following conclusion of a surgical procedure, and are generally removed upon conclusion of the procedure. In many cases, however, it would be desirable to retain the stethoscope tube in place for post-operative monitoring of temperature, heart and lung sounds.

Surgical patients can reduce their risk of certain complications and even death by having an empty stomach. In an elective, non-emergent setting, the patient is instructed to refrain from eating or drinking for several hours prior to surgery to minimize the likelihood of significant gastric content. Emergency surgery, patient non-compliance, and the existence of certain medical conditions, such as diabetes, bowel obstruction, and obesity may increase the likelihood that the patient will have significant gastric contents that may be aspirated into the lungs during or following the surgical procedure.

In order to decrease the risk of such aspiration and avoid the accompanying morbidity, it is possible to introduce a nasogastric tube either prior to or during surgery. The nasogastric tube is equipped with a suction lumen to enable gastric drainage during the surgical procedure. A nasogastric tube may be used in conjunction with an esophageal stethoscope, however this results in crowding of the esophagus.

There have been some attempts to combine stethoscope, temperature monitoring and suction functions in a single esophageal gastric device. Most of these devices are not effectively vented, either because they do not include vent structure, or because such structure is not effective. Attempts to include vent openings in the catheter sidewall of gastric suction devices have not generally been successful because the external apertures are subject to failure when occluded by contact with the interior wall of the stomach. Consequently, the negative pressure or vacuum effect that enables suction drainage of the gastric contents also causes the gastric mucosa to be drawn to the suction openings. These suction openings generally have relatively sharp perimeter margins. Prolonged continuous or repeated intermittent suctioning without adequate venting causes approximation of the gastric mucosa over these margins, resulting in abrasion and eventual erosion of the stomach lining by the vacuum outlet openings of the tube.

There have been some attempts to address this problem by placing external vent openings at a more proximal location in the catheter sidewall, where they will be positioned above the body of the stomach and less subject to occlusion by larger matter in the gastric content. However, such external vent openings remain subject to occlusion and they fail to provide effective venting in the distal area of the tube where the suction openings are located.

Consequently, where it is necessary to continue postoperative monitoring of the patient's core temperature and heart and lung sounds, it is more common to reinsert a dedicated esophageal stethoscope that does not include a suction lumen.

Although both esophageal stethoscopes and nasogastric tubes are frequently employed, introduction of such tubes is associated with risk of injury to the tissues of the nasopharynx, hypopharynx, esophagus, larynx and associated tissues. This risk is enhanced when an esophageal stethoscope is placed for use during surgery and then removed to be replaced by a nasogastric tube during the postoperative recovery period.

Thus, there is a need for a multi-function esophageal gastric tube that may be safely inserted and withdrawn without causing tissue damage, is suitable for extended suctioning use for providing gastric drainage, that includes an effective venting system to prevent damage to the interior surface of the stomach and that also includes structure for monitoring temperature and auscultation that will not be functionally impaired by compression by the surrounding body tissue.

### SUMMARY OF THE INVENTION

The present invention provides a greatly improved esophageal gastric tube having an open proximal end, a closed distal end, and lumens for suction, venting and auscultation. A stethoscope membrane is supported in spaced relation surrounding the tubular sidewall to form a sound chamber. The stethoscope membrane is located for positioning in the mediastinum area of a patient when the tube distal end is inserted into the patient's stomach. The stethoscope lumen extends from the proximal end of the gastric tube to at least the stethoscope membrane for transmitting body sounds. A portion of the tubular sidewall includes a longitudinal slot communicating the sound chamber with the stethoscope lumen, enabling transmission of the patient's heart and lung sounds through the stethoscope membrane and into the lumen for auscultation through acoustical earpieces connected at the proximal end of the lumen. The inner surface of the membrane includes a series of stiffening structures such as circumferential ribs for stiffening the membrane to maintain a preselected spaced relation between the inner surface of the membrane and the slot, so that the membrane will not occlude the slot and will conduct sounds around the perimeter of the tube despite compression of the tube against adjacent body tissues. A thermistor device is positioned distal of the stethoscope lumen for measuring the core temperature of the patient. The suction and vent lumens extend from the proximal end of the gastric tube to its distal end. The distal sidewall of the gastric tube is apertured through to the suction lumen to enable decompression of the stomach and suction drainage of the contents. The vent lumen extends distally to the tip area of the gastric tube, and an internal distal opening permits passage of air from the vent lumen to the suction lumen and serves as a suction break for relieving excessive negative pressure in the stomach of the patient.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a perspective view of an esophageal gastric tube in accordance with the present invention.

FIG. 2 is a greatly enlarged detail of the stethoscope of FIG. 1, with parts broken away to show the circumferential ridges of the stethoscope membrane, details of the thermistor assembly and the slot.

FIG. 3 is a greatly enlarged view of the distal end of the gastric tube of FIG. 1, with the end portion shown in longitudinal section to details of the interior communication between the vent and suction lumens.

FIG. 4 is an enlarged sectional view taken along line 44 of FIG. 1.

FIG. 5 is an enlarged sectional view taken along line 5-5 of FIG. 1.

FIG. 6 is an enlarged transverse section similar to FIGS. 4 and 5, taken below the distal end of the stethoscope membrane, and illustrating a plug effectively terminating the stethoscope lumen.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

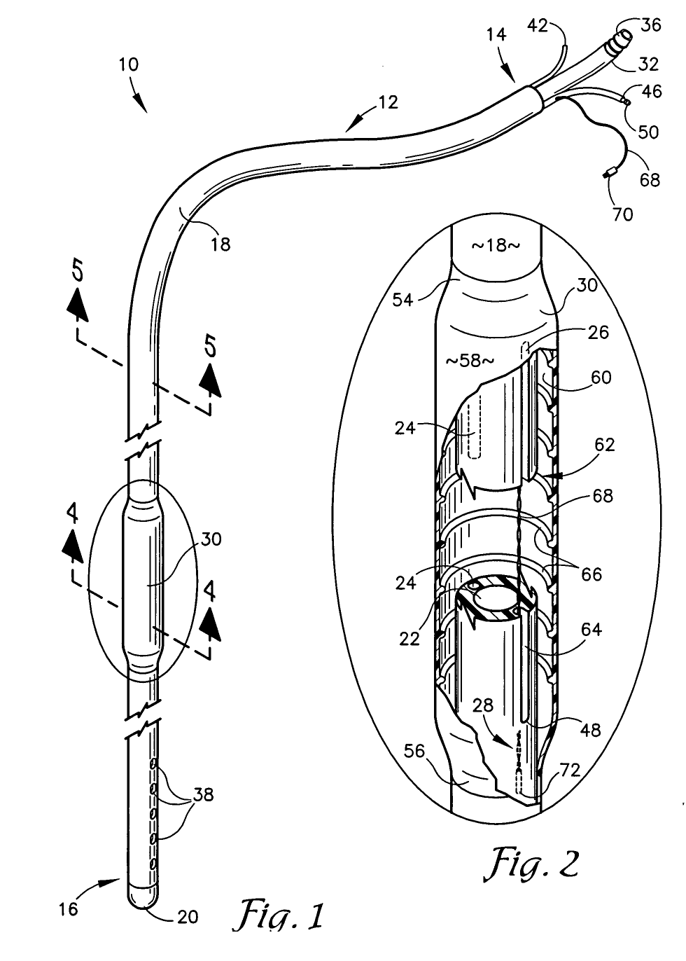

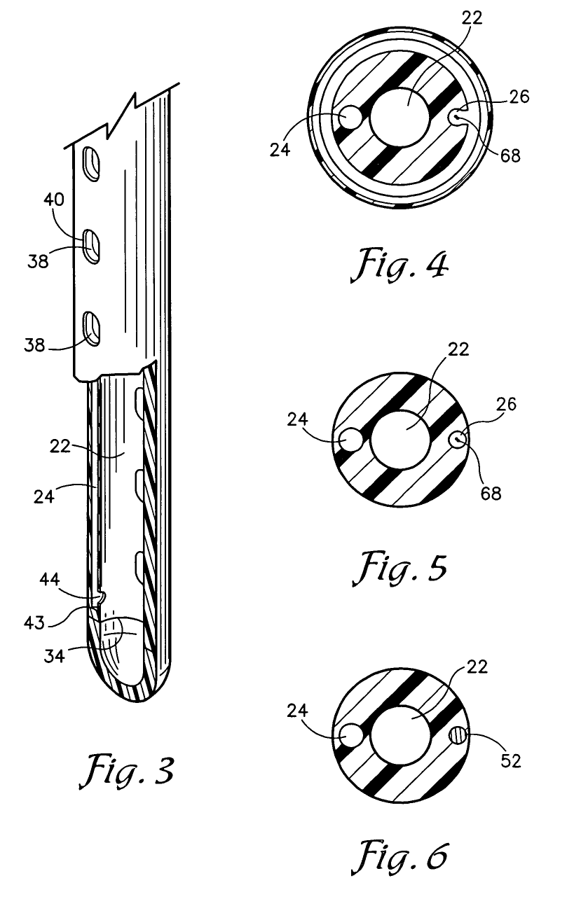

Referring now to the drawing figures, the reference numeral 10 refers to an esophageal gastric tube apparatus in accordance with the invention. As best shown in FIGS. 1 and 3, the gastric tube 10 includes an elongated catheter or tube 12, having a proximal end 14 and a distal end 16 with a connecting sidewall 18. The proximal end 12 is generally open, whereas the distal end 16 is sealed or closed to form a tip 20.

The tube 12 includes a first or suction lumen 22, a second or vent lumen 24 and a third auscultation or stethoscope lumen 26 (FIGS. 1-6). The stethoscope lumen 26 serves as a conduit for a thermistor assembly 28 for monitoring the core temperature of a patient. A portion of the gastric tube sidewall 18 is encircled by a stethoscope pick up or membrane 30 that is located for positioning in the mediastinum area, between the lungs and adjacent the heart, when the distal end 16 of the tube 12 is positioned in the stomach of a patient.

The suction lumen 22 extends axially between the proximal and distal ends 14 and 16 of the tube 12 and includes open proximal and distal ends 32 and 34. The suction lumen proximal end 32 receives a fitting or connector 36 for a suction hose, which turn is connected to a conventional vacuum pump (not shown). The distal end 34 terminates in the area of the tip 20. A series of spaced apart apertures or gastric openings 38 extend between the distal portion of the tubular sidewall 18 and the suction lumen 22 to enable decompression of the stomach and passage of fluids and materials from the stomach into the suction lumen 22.

The gastric openings 38 extend at spaced intervals around the tubular sidewall 18 to enable multidirectional suctioning and to ensure access in the event of a blockage or occlusion of one side of the tube 12. The apertures 38 each include an external perimeter margin or edge 40 that is generally relieved, with the angle being beveled, chamfered or otherwise radiused to enable smooth, sliding passage of the tube 12 over the mucosa and past the nasal turbinates. This configuration reduces “grabbing” of the turbinates by sharp edges of the openings 38 and reduces trauma during withdrawal of the tube 12, such as, for example, abrasion and bleeding.

The vent lumen 24 also extends between the between the proximal and distal ends 14 and 16 of the tube 12 in substantially parallel side-by-side relation with the suction lumen 24. The vent lumen 24 includes open proximal and distal ends 42 and 43, with the distal end 43 terminating in the area of the tip 20. It is foreseen that the proximal opening of the vent 42 may be equipped with a check valve or selectively permeable membrane that permits gases such as air but not liquids to enter into the lumen 24. The vent distal end 43 and suction lumen distal end 34 include an opening or passageway 44 therebetween for permitting internal passage of ambient air within the tube 12 from the vent lumen 24 into to the suction lumen 22. Depending on the construction of the tip 20, the tip itself may serve as the passageway 44 for air between the open ends of the suction and vent lumens 22 and 24. It is also foreseen that a passageway 44 may be provided by relieving a portion of the respective lumen sidewalls adjacent the distal end 34 and 43.

The stethoscope lumen 26 extends from the proximal end 14 of the tube 12 to at least the distal portion of the stethoscope membrane 30. The stethoscope lumen 26 includes an open proximal end 46 and a closed distal end 48. The proximal end 46 receives a connector 50, such as a male Luer fitting, for connection with a conventional acoustical ear piece (not shown). For ease of manufacture, the stethoscope lumen 26 may be constructed to extend all the way to the distal end 16 of the tube 12. In that case, an elongated seal or plug 52 serves to terminate the auscultatory portion of the lumen 26. Termination of the lumen 26 or its auscultatory portion in the vicinity of the stethoscope membrane 30 provides for spatial separation between the stethoscope portion of the gastric tube 10 and the noisier suction and vent portions of the device. This reduces transmission of the suction sounds to the stethoscope portion of the device.

The stethoscope membrane 30 encircles the tubular sidewall 18 in surrounding relation to an intermediate portion of the tube 12 that is positioned in the area of the mediastinum during use. The membrane 30 includes proximal and distal ends, 54 and 56, that are sealed, as by heat or fusion welding, to the outer surface of the sidewall 18. The membrane 30 also includes an outer surface 58 and an inner surface 60, which is radially spaced from the tube sidewall 18 to form a sound chamber 62 therebetween. The tubular sidewall 18 includes an elongate, longitudinal channel or slot 64 that extends axially into the stethoscope lumen 26 for enabling transmission of sound between the surrounding sound chamber 62 and the stethoscope lumen 26. The slot extends substantially the full length of the sound chamber formed by the stethoscope membrane 30, enabling transmission of all of the sounds picked up by the membrane 30 into the slot 64. It is foreseen that the slot 64 may also be constructed as a series of discontinuous slots or holes extending substantially the full length of the sound chamber. These slots or holes may be elongated, round or of any other suitable configuration or combination thereof.

The inner surface 60 of the stethoscope membrane 30 includes a series of inwardly projecting stiffening structures, supports or ribs 66 that serve to provide stiffness and radial support to the membrane 30 while maintaining a selected spacing or spaced relationship between the inner surface 60 of the membrane 30 and the sidewall 18. The ribs 66 provide intermediate support for the tent-like arrangement of the stethoscope membrane 30 that forms the sound chamber 62, prevent collapse of portions of the chamber 62 and protect the slot 64 from blockage or occlusion by the membrane 30. When surrounding tissues compress the stethoscope membrane 30 toward the tube 12, the ribs 66 support the membrane 30 in spaced relation to the sidewall 18 to form a series of circumferential channels within the sound chamber 62 for transmitting sound from around the entire perimeter of the membrane to the slot 64.

While the stiffening structures or ribs 66 are depicted in FIG. 2 as being of spaced circumferential construction, they may also be of spaced, generally helical or spiral construction or they may comprise a series of short, spaced ribs, cones, rounded or flat-topped bumps, multilateral, or any other suitable shape or combination of shapes that permits transmission of sounds 360° around the circumference of the tubular sidewall 18 to the slot 64 and then into the stethoscope lumen 26 for enabling substantially omnidirectional or 360° auscultation of body sounds around the tube 12. Those skilled in the art will appreciate that the supports 66 may alternatively be attached to the outer surface of the sidewall 18, in which case some supports or portions thereof may extend over one or more portions of the slot 64. It is also foreseen that the outer surface of the tubular sidewall 18 may include a series of channels, grooves or other pattern of perforations that function in similar fashion to the ribs 66 to permit transmission of sound from body areas adjacent the full perimeter of the stethoscope membrane 30 to the slot 64.

The thermistor assembly 28 includes a pair of electrical conductors 68 that extend distally through the proximal opening 46 of the stethoscope lumen 26. The proximal ends of the conductors 68 are joined to an electrical connector or fitting 70 for connection with a conventional temperature monitoring device. The distal ends of the conductors 68 are joined to a temperature sensing device or thermistor 72, positioned distally of the stethoscope lumen 26 and embedded in the sidewall 18 of the tube 12. Where the stethoscope lumen 26 extends to the distal end 16 of the tube 12, the thermistor 72 may be embedded within the plug 52, or positioned distally of the plug 52. The electrical conductors 68 are of a relatively fine gauge so that they do not occlude the stethoscope lumen 26. The thermistor 72 is positioned distal to the stethoscope lumen 26 so that it does not occlude the lumen and so that the air within the lumen does not act to insulate the temperature sensing device.

The tube 12 is constructed of an inert, flexible synthetic resinous material suitable for medical use inside the body and of a length suitable for placement of the tip 20 in the stomach of a patient. The tube 12 may be of unitary construction, with the lumens 22, 24 and 26 formed by extrusion, boring or other suitable means. Alternatively, a series of separate tubes may be bundled together and encased within an external sheath or sidewall 18. The sidewall 18 may also include radiopaque or visually discernable markings to assist in placement or subsequent location of various portions of the device 10 by imaging.

The tube 12 is generally constructed so that the suction lumen 22 is somewhat larger in diameter than the vent lumen 24 and the stethoscope lumen 26, although the size of each lumen may be vary. While the lumens 22, 24 and 26 are depicted in the drawing figures as each having a generally circular cross sectional aspect, it is also foreseen that they may have ovoid, polygonal, a combination of lateral and curvate, cruciform or any other suitable geometric configuration. The suction lumen 22 is depicted as axial, with the vent and stethoscope lumens 24 and 26 in opposed, side-by-side relation. It is also foreseen that the vent and stethoscope lumens 24 and 26 may be positioned adjacent each other or at any other spaced apart position and the suction lumen 22 may be positioned off the center axis.

The tip 20 may be of integral construction with the tube 12, or it may be fabricated separately and fused to the distal end 16 of the tube by any suitable means. The tip 20 is depicted as generally hollow in FIG. 3. A solid or substantially solid atraumatic tip 20 fabricated from a soft, elastomeric synthetic resin material may also be employed for cushioning against trauma during vigorous insertion of the device 10. The proximal end of the tip 20 may also include a hollow area to permit passage of air from the distal end of the vent lumen 43 to the distal end of the suction lumen 34.

The stethoscope membrane 30 is formed of a thin layer of a synthetic resin material that is sealed at the proximal and distal ends 54 and 56 to the external surface of the tubular sidewall 18, as by fusion welding or heat sealing. It is foreseen that a transducer such as a microphonic element may be substituted for the stethoscope membrane 30 and connected with appropriate electrical conductors in a manner similar to the thermistor 72.

The device 10 may also be constructed to include one or more additional sensing devices for monitoring of tissue gas and pH, as well as ECG and cardiac pacing. It may also be constructed to exclude one or more of the sensing devices, such as the thermistor assembly 28.

In use, a physician or other suitably trained user inserts the tip 20 of the esophageal gastric tube 10 into the nose or mouth of a patient. The user urges the tube 10 distally through the nasopharynx or pharynx, into the esophagus and further distally until the distal end 16 is positioned within the stomach and the stethoscope membrane 30 is positioned in the mediastinum area adjacent the heart. The radiused margins 40 of the suction openings 38 in the sidewall 18 serve to protect against abrasion of the nasal turbinates and mucosal surfaces which would normally occur during insertion of a conventional nasogastric tube.

Once the tube 12 is properly positioned for suction of the gastric contents and auscultation of body sounds, connections are made between the suction connector 36, vent proximal end 42, stethoscope connector 50 and thermistor electrical connector 70 and a respective pump, check valve or membrane, ear piece and temperature monitor. Actuation of the pump produces suction pressure which decompresses the stomach and commences evacuation of the gastric contents. While the pump may be actuated intermittently, entry of ambient air through the vent lumen 24 prevents excessive suction and makes this manner of operation unnecessary for protection of the gastric mucosa. The gastric contents pass through the distal apertures 38, into the suction lumen 22, and travel proximally through the lumen to a remote trap. Actuation of the temperature monitoring device enables an electrical current to travel along the conductors 68 within the stethoscope lumen 26 to the distal thermistor 72. The thermistor 72 continuously measures the core body temperature of the patient and transmits the information via the conductors 68 to compatible meter circuitry (not shown) and a remote monitor display.

The stethoscope membrane 30 functions as a diaphragm, so that body sounds are transmitted pneumatically from the sound chamber 62 formed by the stethoscope membrane 30 and through the stethoscope lumen 26 to the ear of a remote listener. Sounds are transmitted from the entire circumference of the membrane 30 even when the membrane is compressed against the sidewall 18 because the circumferential ribs 66 support the membrane 30 in outstanding relation to the sidewall to form channels within the sound chamber 62. Sounds are transmitted within the sound chamber 62 to the slot 66 and then proximally and outwardly through the stethoscope lumen 26 to the ear of a listener. The circumferential arrangement of the ribs 66 ensures that sound will be transmitted from all sides of the stethoscope membrane 30 to the slot 64, even if a portion of the membrane becomes compressed against the sidewall 18.

Advantageously, the combination of the stethoscope lumen 26 with the slot 64 and the rib supports 66 on the stethoscope membrane 30 cooperatively enable 360° auscultation of body sounds without interruption or diminution of sound quality when the stethoscope membrane 30 is subject to compressive pressure from adjacent tissues as may occur during a surgical procedure. The spaced relationship or spacing between the suction lumen opening 34 and the sound chamber 62 permits auscultation while reducing interruption by noise from suction drainage. The distal positioning of the vent opening 44 provides effective venting. The internal arrangement of the passageway 44 from the vent distal end 43 to the suction lumen distal end 34 within the enclosed tubular sidewall 18 and tip 20 in combination with the beveled suction apertures enables placement and retention of the device in the body of a patient for an extended period of time while reducing the risk of abrasion and ulceration of the gastric mucosa that is generally associated with continuous, long term gastric suction.

It is to be understood that while certain forms of the present invention have been illustrated and described herein, it is not to be limited to the specific forms or arrangement of parts described and shown.

Having described the preferred embodiments of the present invention, the following is claimed as new and desired to be secured by Letters Patent:

1. An esophageal gastric tube for insertion through the esophagus and into the stomach of a patient and comprising:
a. an elongated flexible tube having a proximal end, a distal end and a tubular sidewall therebetween;
b. a stethoscope membrane encircling a portion of said sidewall, the membrane being located along said tube to enable positioning in a mediastinum area of a patient when the tube distal end is inserted into the patient's stomach, said membrane having at least a portion radially spaced from said sidewall to form a sound chamber therewith;
c. a first lumen extending between said tube proximal end and said tube distal end and including means for connection of said first lumen with a suction device at said proximal end, said tubular sidewall including a gastric opening communicating with said first lumen, enabling suctioning of fluids into said first lumen;
d. a second lumen extending between said tube proximal end and said tube distal end, said second lumen including a distal opening conducting air from said tube proximal end and into said first lumen for limiting suction pressure at said gastric opening;
e. a third lumen extending from said tube proximal end at least to said stethoscope membrane for transmitting body sounds from said membrane to a stethoscope; and
f. a portion of said tubular sidewall encircled by said stethoscope membrane including a longitudinal slot communicating said sound chamber with said third lumen, enabling transmission of body sounds through said stethoscope membrane and into said third lumen for proximal auscultation.

2. The esophageal gastric tube as set forth in claim 1:
a. said gastric tube distal end further including an enclosed tip; and
b. said first and second lumens each having a respective open distal end adjacent said tip, enabling passage of air from the end of said second lumen within the enclosed tip of said tube and into the end of said first lumen for relieving suction pressure at said gastric opening.

3. The esophageal gastric tube as set forth in claim 1, wherein:
a. said stethoscope membrane includes an outer surface and an inner surface; and
b. said inner surface includes a plurality of stiffening structures for maintaining a preselected space between said stethoscope membrane inner surface and said tubular sidewall, enabling substantially omnidirectional auscultation with said sound chamber.

4. The esophageal gastric tube as set forth in claim 1, wherein said first lumen communicates with a plurality of gastric openings through said tubular sidewall enabling rapid suctioning of fluids into said first lumen.

5. The esophageal gastric tube as set forth in claim 4, wherein said gastric openings each include an external relieved perimeter margin for enabling sliding passage of said gastric openings over a mucosal surface with reduced likelihood of trauma.

6. The esophageal gastric tube as set forth in claim 1, wherein said first, second and third lumens of said gastric tube are disposed in side-by-side relation.

7. The esophageal gastric tube as set forth in claim 1, and including:
a. said third lumen extending between said tube proximal end and said tube distal end; and
b. a plug positioned within said third lumen in spaced relation to said stethoscope membrane for enabling transmission of body sounds through said stethoscope membrane and into said third lumen for proximal auscultation while blocking out sounds from said distal ends of said first and second lumens.

8. An esophageal gastric tube for insertion through the esophagus and into the stomach of a patient and comprising:
a. an elongated flexible tube having a proximal end, a distal end and a tubular sidewall therebetween;
b. a stethoscope membrane encircling a portion of said sidewall, the membrane being located along said tube to enable positioning in a mediastinum area of a patient when the tube distal end is inserted into the patient's stomach, said membrane having at least a portion radially spaced form said sidewall to form a sound chamber therewith;
c. a first lumen extending between said tube proximal end and said tube distal end and including means for connection of said first lumen with a suction device, said tubular sidewall including a gastric opening communicating with said first lumen, enabling suctioning of fluids into said first lumen;
d. a second lumen extending between said tube proximal end and said tube distal end, said second lumen including a distal opening for conducting air from said tube proximal end and into said first lumen for limiting suction pressure at said gastric opening;
e. a third lumen extending from said tube proximal end at least to said stethoscope membrane for transmitting body sounds from said membrane to a stethoscope;
f. said third lumen including a proximal end and a distal end;
g. a thermistor positioned within said gastric tube in spaced relation to said third lumen for measuring a core temperature of the patient; and
h. a portion of said tubular sidewall encircled by said stethoscope membrane including a longitudinal slot communicating said sound chamber with said third lumen, enabling transmission of body sounds through the stethoscope membrane and into said third lumen for auscultation.

9. The esophageal gastric tube as set forth in claim 8:
a. said gastric tube distal end further including an enclosed tip; and
b. said first and second lumens each having a respective open distal end adjacent said tip, enabling passage of air from the end of said second lumen within the enclosed tip of said tube and into the end of said first lumen for relieving suction pressure at said gastric opening.

10. The esophageal gastric tube as set forth in claim 8, wherein:
a. said stethoscope membrane includes an outer surface and an inner surface; and
b. said inner surface includes a plurality of circumferential ribs for maintaining a preselected space between said stethoscope membrane inner surface and said tubular sidewall, enabling substantially omnidirectional auscultation with said sound chamber.

11. The esophageal gastric tube as set forth in claim 8, wherein said first lumen communicates with a plurality of gastric openings through said tubular sidewall enabling rapid suctioning of fluids into said first lumen.

12. The esophageal gastric tube as set forth in claim 11, wherein said gastric openings each include an external relieved perimeter margin for enabling sliding passage of said gastric openings over a mucosal surface.

13. The esophageal gastric tube as set forth in claim 8, wherein said first, second and third lumens of said gastric tube are disposed in substantially parallel relation.

14. The esophageal gastric tube as set forth in claim 8, wherein said thermistor is positioned distally of said third lumen distal end for measuring a core temperature of the patient while optimizing transmission of body sounds through said third lumen.

15. The esophageal gastric tube as set forth in claim 8, and including:
a. said third lumen extending between said tube proximal end and said tube distal end; and
b. a plug positioned within said third lumen in spaced relation to said stethoscope membrane for enabling transmission of body sounds through said stethoscope membrane and into said third lumen for proximal auscultation while blocking out sounds from said distal ends of said first and second lumens.

16. An esophageal gastric tube for insertion through the esophagus and into the stomach of a patient and comprising:
a. an elongated flexible tube having a proximal end, a distal end and a tubular sidewall therebetween;
b. a stethoscope membrane encircling a portion of said sidewall, the membrane being located along said tube to enable positioning in a mediastinum area of a patient when the tube distal end is inserted into the patient's stomach, said membrane being radially spaced from said sidewall to form a sound chamber therewith;
c. said stethoscope membrane includes an outer surface and an inner surface;
d. said inner surface includes a plurality of circumferential ribs for maintaining a preselected space between said stethoscope membrane inner surface and said tubular sidewall, enabling omnidirectional auscultation with said sound chamber;
e. a first lumen extending between said tube proximal end and said tube distal end and including means for connection of said first lumen with a suction device at said proximal end, said tubular sidewall including a gastric opening connecting with said first lumen, enabling suctioning of fluids into said first lumen;
f. a second lumen extending between said tube proximal end and said tube distal end, said second lumen including a distal opening for conducting air from said tube proximal end and into said first lumen for limiting suction pressure at said gastric opening;
g. a third lumen extending from said tube proximal end at least to said stethoscope membrane for transmitting body sounds from said sound chamber to a stethoscope;
h. a portion of said tubular sidewall encircled by said stethoscope membrane including a longitudinal slot communicating with said third lumen, enabling transmission of body sounds through the stethoscope membrane and into said third lumen for auscultation;
i. and first second and third lumens disposed in substantially parallel side-by-side relation;
j. said third lumen includes a proximal end and a distal end;
k. a thermistor positioned within said gastric tube in spaced relation to said third lumen for measuring a core temperature of the patient; and
l. said tube distal end further includes an enclosed tip enabling the passage of air from the end of said second lumen into the end of said first lumen for relieving suction pressure.

17. The esophageal gastric tube as set forth in claim 16, wherein:
a. said thermistor is positioned within said gastric tube distally of said third lumen distal end.
